(19) United States
(12) Patent Application Publication
(10) Pub. No.: US 2023/0041873 A1
(43) Pub. Date: Feb. 9, 2023

(54) Device For Measuring The Turbidity Of Cerebrospinal Fluid And Associated Method

(71) Applicant: Neurallys, Paris (FR)

(72) Inventors: Philippe Auvray, Caen (FR); Johan Lazardeux, Fresne La Mere (FR)

(73) Assignee: Neurallys, Paris (FR)

(21) Appl. No.: 17/790,003

(22) PCT Filed: Dec. 30, 2020
(86) PCT No.: PCT/FR2020/052642

§ 371 Date: Jun. 29, 2022

(30) Foreign Application Priority Data
Dec. 30, 2019 (FR) 1915742

(51) Int. Cl. A61B 5/145; A61B 5/1455; A61B 5/00; G01N 21/53

(52) U.S. Cl. CPC A61B 5/14507; A61B 5/686; A61B 5/1455; A61B 5/4058; G01N 21/53

(57) ABSTRACT

A device for measuring the turbidity of cerebrospinal fluid includes, a source of a light signal comprising having one or more wavelength(s), such that at least part of the emitted light signal passes through the cerebrospinal fluid; a flow element including an inlet and an outlet, the flow element being suitable for allowing cerebrospinal fluid to flow between the inlet and the outlet; an opaque element, arranged to absorb at least part of the emitted light signal after it has passed through the cerebrospinal fluid, and to allow another part of the emitted light signal to be reflected after it has passed through the cerebrospinal fluid; and an optical detector configured to detect the light signal after it has passed through the cerebrospinal fluid.

### FIELD OF THE INVENTION

The present invention concerns the measurement of cerebrospinal fluid turbidity. The invention also concerns an associated system and method.

### STATE OF THE ART

Devices for measurement of ambulatory intracranial pressure for patients with hydrocephalus and bearing a ventriculoperitoneal or ventriculoatrial shunt system and comprising a valve are known.

The surgery for implanting such a valve or revision of such a valve has high risks for the patient, for example infectious risks. Such surgery therefore constitutes a risky situation for the patient. An infection can actually result in meningitis. It is then necessary to remove the system from the patient. A powerful and long antibiotic treatment is then necessary before being able to reimplant a new shunt system.

An infection can be detected by means of a puncture, for example in a reservoir associated with the valve if the valve is equipped with one, to take a sample of the cerebrospinal fluid. Observing such fluid with the naked eye makes it possible to quickly provide preliminary information regarding the presence of an ongoing infection. A bacteriological analysis is also done in a medical laboratory. However, these sampling methods have a non-zero infectious risk by the puncture, while in some cases, no infection was present beforehand. Often, the infection is detected late, generating serious risks for the patient and very difficult care. It is therefore problematic to implement them regularly, which limits the chances of quickly and preventatively detecting an infection resulting from the valve implant surgery itself or an infection with other origins.

### DISCLOSURE OF THE INVENTION

One objective of the invention is to alleviate these disadvantages. One objective of the invention is, in particular, to allow rapid detection of an infection.

According to a first aspect of the invention, a device for measuring the turbidity of cerebrospinal fluid is proposed for this purpose, comprising:
a source able to emit a light signal comprising one or more wavelengths, such that at least a part of the emitted light signal passes through the cerebrospinal fluid,
a flow element comprising an inlet and an outlet, the flow element being suitable for allowing the cerebrospinal fluid to circulate between the inlet and the outlet,
an opaque element, arranged to absorb at least a part of the emitted light signal after it has passed through the cerebrospinal fluid, and to reflect another part of the emitted light signal after it has passed through the cerebrospinal fluid,
an optical detector configured to detect the light signal after it has passed through the cerebrospinal fluid.

The invention also comprises the following characteristics, taken alone or according to any one of the technically-possible combinations thereof:
the at least one opaque element extends around at least one portion of the flow element,
the at least one opaque element comprises a light ray absorption zone with an absorbance coefficient greater than or equal to 1 CU,
the flow element comprises a wall transparent or translucent to the wavelengths of the light signals emitted by the source, preferably having a transmission coefficient greater than 80%,
the flow element and/or the opaque element comprises an opening opposite which the source and/or the detector can be positioned,
the light signals emitted by the source comprise one or more wavelength(s) comprised between 300 nm and 850 nm,
the source comprises one or more light-emitting diode(s), for example three light-emitting diodes, allowing each one to generate a light signal of wavelength different from the light signal or signals generated by the other diode(s),
the detector is suitable for measuring several ranges of disjoint wavelengths,
the detector is suitable for detecting a light signal comprising one or more wavelength(s) comprised between 300 nm and 1000 nm,
means for measuring pressure, for example of cerebrospinal fluid.

The invention also concerns an implant comprising a device (10) according to any one of the preceding claims.

The invention also concerns a method for measuring the turbidity of cerebrospinal fluid by means of such a device or such an implant, comprising the steps of:
circulating the cerebrospinal fluid through the flow element,
emitting a light signal by the source,
reflecting by the flow element and/or the opaque element of the light signal emitted after at least a first passage through the cerebrospinal fluid present in the flow element,
detecting, by the measurement means, of the light signal reflected after at least a second passage through the cerebrospinal fluid present in the flow element.

The method also concerns a method for manufacturing such a device or such an implant, comprising a step of obtaining the opaque element at the level of at least a part of the flow element, the obtaining step comprising, for example, injecting coloured pigment of the opaque element around the flow element.

### DESCRIPTION OF THE FIGURES

Other characteristics, objectives and advantages of the invention will appear from the following description, which is purely illustrative and non-limiting and should be read with regard to the attached drawings, in which:

FIG. 1a schematically illustrates a device according to one example of embodiment of the invention.

FIG. 1b schematically illustrates a device according to another example of embodiment of the invention.

FIG. 1c schematically illustrates an opening of the device of FIGS. 1a or 1c according to one example of embodiment of the invention.

FIG. 1d illustrates a sectional view of the opening of FIG. 1c along plane A-A.

FIG. 1e schematically illustrates an opening of the device of FIGS. 1a or 1c according to another example of embodiment of the invention.

FIG. 1f illustrates a sectional view of the opening of FIG. 1c along plane B-B.

FIG. 1g schematically illustrates a module of the device of FIGS. 1a or 1c.

FIG. 1h schematically illustrates an assembly of the device of FIGS. 1a or 1c.

FIG. 2 schematically illustrates an implant according to one example of embodiment of the invention.

FIG. 3 schematically illustrates a measurement method according to one example of embodiment of the invention.

FIG. 4 schematically illustrates a manufacturing method according to one example of embodiment of the invention.

FIG. 5 schematically illustrates a system according to one example of embodiment of the invention.

FIG. 6a schematically illustrates the molar extinction spectra of different chromophores.

FIG. 6b schematically illustrates the absorption coefficient of water as a function of wavelength.

FIG. 6c schematically illustrates the absorption coefficient of a subarachnoid haemorrhage as a function of wavelength.

FIG. 6d schematically illustrates the ratio of light intensities by the measurement means of a device according to an example of embodiment of several liquids relative to pure water.

Throughout the figures, similar elements bear identical references.

### DETAILED DESCRIPTION OF THE INVENTION

### Turbidity Measuring Device

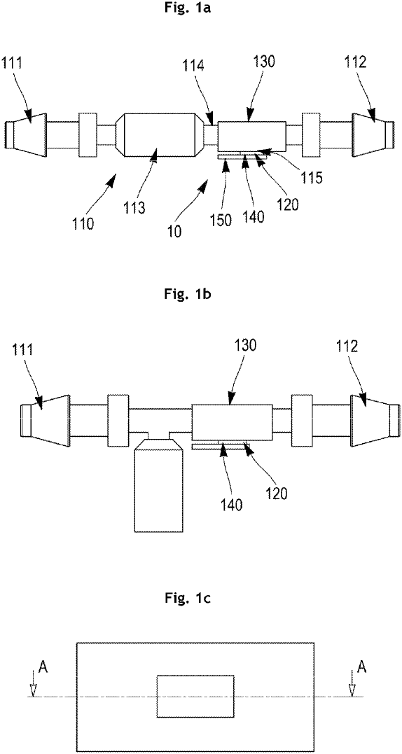

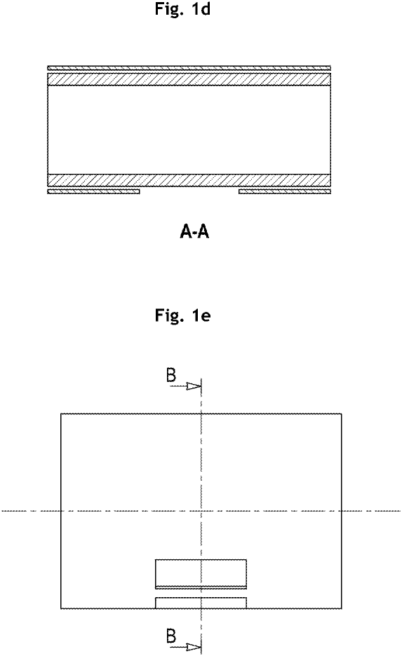

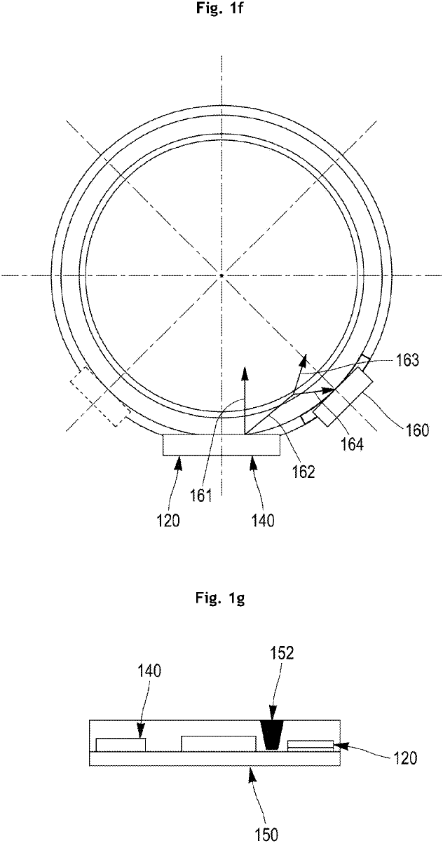

In reference to FIGS. 1a, 1b, 1c, 1d, 1e and 1f, a device 10 is described for measuring the turbidity of cerebrospinal fluid.

The device 10 comprises the emission means 120 of a light signal comprising one or more wavelength(s), for example a source capable of emitting a light signal comprising one or more wavelength(s), so that at least a part of the light signal emitted passes through the cerebrospinal fluid.

The device 10 comprises a flow element 110 comprising an inlet and an outlet. The flow element 110 is designed to allow the circulation of cerebrospinal fluid between the inlet and the outlet.

The device 10 comprises an element 130. The element 130 is, for example, an opaque element, for example opaque to the wavelength(s) of the light signal emitted by the source, for example contrasting, for example absorption and/or reflection. The opaque element 130 is arranged, for example disposed at the level of at least a part of the flow element, to absorb at least a part of the light signal emitted after passage through the cerebrospinal fluid, for example after a first passage through the cerebrospinal fluid, and to reflect another part of the light signal emitted after passage through the cerebrospinal fluid, for example a first and/or second passage through the cerebrospinal fluid, for example the part of the flow element and the opaque element also being suitable to allow the reflection of another part of the light signal emitted after passage through the cerebrospinal fluid.

The device 10 comprises optical detection means 140, for example a detector, configured to detect the light signals, for example reflected, after passage through the cerebrospinal fluid.

It is thus possible to obtain a reliable measurement of the cerebrospinal fluid.

During its passages through the cerebrospinal fluid, the light signal emitted then optionally reflected experiences absorption and diffusion phenomena that modify it. For example, if the cerebrospinal fluid is bloody, therefore reddish coloured, the optical signal will be mainly absorbed by the fluid at certain characteristic wavelengths. For example, if the fluid is infectious, in other words whitish, this fluid will diffuse the light signal that passes through it.

It is thus possible to obtain in situ a reliable measurement of the cerebrospinal fluid turbidity. Such a device makes it possible to regularly perform measurements, especially in the weeks following surgery, for example valve surgery, which is typically the period of major infectious risk.

The device is, for example, suitable for detecting a leukocyte density greater than 500 elements/mm³ in cerebrospinal fluid. Indeed, when the cerebrospinal fluid becomes infectious, the turbidity of the cerebrospinal fluid is modified in proportion to the increased density of leukocytes in the cerebrospinal fluid.

The device is, for example, suitable for detecting a haemoglobin and/or oxyhaemoglobin and/or methaemoglobin and/or bilirubin density greater than 1000 elements/mm³ in cerebrospinal fluid. Indeed, in the case of bleeding, for example, the colour of the cerebrospinal fluid can also be modified by the presence of haemoglobin and/or oxyhaemoglobin and/or methaemoglobin and/or bilirubin. Bilirubin is for example linked to the degeneration of red blood cells in the cerebrospinal fluid.

### Flow Element

The flow element 110 can be a channel. The flow element 110 can comprise a wall 114 transparent or translucent, for example, to the wavelength(s) of the light signal emitted by the source 120. The flow element 110 is transparent or translucent, for example. The transparent or translucent wall can have a transmission coefficient preferably greater than 80%, for example at the wavelength(s) of the light signal emitted by the source. The transparency coefficient can be the ratio between the light intensity transmitted and the light intensity received, for example by the wall.

The flow element 110 and/or the opaque element can comprise one or more openings 115 opposite which, for example at the level of which, the source and/or the detector can be positioned. The source and the detector can be positioned at the same opening such as illustrated in FIG. 1c and FIG. 1d. Alternatively or in complement, the source and the detector can be positioned at two different openings such as illustrated in FIG. 1c and FIG. 1d, for example two openings offset from one another, for example offset radially relative to the flow element. It is thus possible to detect a possible deposit at the flow element and thus avoid obtaining false positives.

The flow element 110 and/or the wall 114, can comprise or be made of a transparent or translucent material, for example, to the wavelength(s) of the light signal emitted by the source. The material can be or comprise silicone, for example implantable silicone, and/or a polycarbonate, for example, a urethane polycarbonate, and/or a polymer, for example a methyl polymethacrylate or a cycloolefin copolymer.

The flow element 110 can comprises an inlet 111 and an outlet 112. The inlet 111 is designed to allow the entrance of cerebrospinal fluid into the flow element. The outlet 112 is designed to allow the exit of cerebrospinal fluid from the flow element 110.

At the inlet 111 and/or the outlet 112, the flow element can comprise a connector. The connector can be a connector for connection to a ventricular catheter. Alternatively, the connector can be a connector for connection to a control valve or means for fluid communication with a control valve.

The flow element 110 can comprise or be a catheter, for example a flexible or rigid catheter, and/or a chamber 1131 and/or a valve. The chamber 1131 is, for example, a pressure measurement chamber, for example for cerebrospinal fluid.

The flow element 110 can comprise an opening 115, for example a window, at which is or are fixed the source 120 and/or the reception means 140.

### Source

The source 120 can comprise one or light emitters.

The source 120 can be a source capable of emitting a light signal in the visible spectrum. The light signal emitted can comprise one or more wavelength(s). The source 120 can be designed to emit at least one light signal comprising one or more wavelength(s) comprised between 300 nm and 850 nm,

The source 120 can comprise one or more light-emitting diode(s), for example three or more light-emitting diodes. Each light-emitting diode can make it possible to generate a different light signal from that of the other light-emitting diode(s), for example a light signal of different wavelength from the light signal or signals generated by the other diode(s).

The source 120 can comprise three light-emitting diodes each able to emit a primary colour, for example red and/or green and /or blue.

The source 120 can be disposed upstream or downstream relative to the detector 140. Upstream or downstream means upstream or downstream in the direction of flow of cerebrospinal fluid in the flow element.

In reference to FIG. 1g, a light barrier 152 can be formed between the emission means, for example the source 120 and the detection means, for example the detector 140. Such a light barrier 152 makes it possible to limit the detection of light signals emitted by the source 120 but which would not have passed through the cerebrospinal fluid and/or would not have been reflected by the opaque element.

### Detector

The detector 140 can be designed to measure the colour level in the cerebrospinal fluid.

The detector 140 can be designed to detect a light signal comprising one or more wavelength(s) comprised between 300 nm and 1000 nm.

The detector 140 can comprise or be a sensor, for example a colour sensor, for example for red and/or green and/or blue colours.

The detector 140 can be designed to measure one or more areas of disjoint wavelengths, each area belonging, for example, to the visible and/or invisible spectrum, for example to the infrared.

The detector 140 can comprise one or more light detectors, for example sensors. The detector 140 can comprise one or more photosensitive surface(s).

In reference to FIG. 1f, the device can also comprise detection means, for example a detector 160, for example additional, of a deposit, for example a lipid or cellular deposit, for example at the inner wall of the device. The detector 160 is, for example, disposed at another opening. The detector 160 is, for example, configured to detect the reflected part 162 of an incident wave 161 issued from the source 120 and which, contrary to the diffracted part 163, is reflected against the inner wall of the device due to the presence of a deposit. It is thus possible to detect the presence of a deposit that can result in false positives and/or to correct a defect due to the deposit, especially by processing the measurement performed by the detector 120 by the measurement performed by the deposit detector 160, for example via processing means such as described below.

### Assembly

The device can comprise an assembly 150, for example an optical assembly. In reference to FIG. 1h, the assembly 150 is described. The assembly can comprise, for example integrate, the source 120 and the detector 140, for example an optical module comprising the source 120 and the detector. The module can comprise the light barrier 152. The assembly can comprise a support 151, on which are positioned, for example fixed, the source 120 and/or the detector 140, for example the module. The support is or comprises, for example, an electronic card.

The assembly 150 or the module can have a length comprised between 3 and 10 mm, for example between 5 and 6 mm. The assembly 150 or the module can have a width comprised between 1 and 3 mm, for example between 1.5 and 2 mm. The assembly or the module 150 can have a thickness comprised between 0.5 and 2 mm, for example between 0.5 and 1.5 mm.

The module forms, for example, a sensor, for example a sensor of the P12347-01CT type sold by Hamamatsu.

The assembly 150 and/or the module can be disposed at the opening 115. The assembly 150 and/or the module can be fixed to the flow element 110, for example, at the opening 115.

The assembly 150 can comprise control means. The assembly 150 or the control means can comprise a control unit 153. The control unit 153 can be designed to control the source 120 and/or the reception means 140. The assembly 150 or the control means can comprise a microcontroller 154, for example, configured to communicate with the control unit 153. The assembly 150 can comprise transmission means 155, comprising, for example, a transmitter, for example a radiofrequency transmitter.

### Opaque Element

In addition to allowing the reflection of the emitted light signal, the opaque element can also provide a contrast, in particular in the case of a whitish cerebrospinal fluid characteristic of an infection, or red characteristic of a haemorrhage.

The opaque element extends, for example, around at least one portion of the flow element. Alternatively, the opaque element can extend along an inner wall of at least one portion of the flow element, the portion of the flow element extending around the opaque element.

The at least one opaque element 130 can be or comprise at least one layer, for example opaque. The at least one opaque element 130, for example the layer, can be or comprise at least one coating, for example opaque. The at least one opaque element 130, for example the layer or the coating, can be or comprise at least one sheath, for example opaque, for example a sheath for example formed of or comprising implantable silicone filled with coloured pigments, for example black pigments.

The at least one opaque element 130 can comprise a black and/or matte light signal reflection surface. The opaque element can be black in colour.

The opaque element 130 forms, for example, an opaque background, for example a monochrome opaque background.

The at least one opaque element 130 can comprise at least one light ray absorption zone with an absorbance coefficient greater than or equal to 1 CU (concentration unit).

The opaque element is, for example a high-contrast opaque element.

### Pressure Measurement Means

The device can also comprise the pressure measurement means 113, for example for intracranial and/or cerebrospinal fluid pressure. The pressure measurement means 113 are or form, for example, an element for measuring pressure. The pressure measurement means 113 can comprise the pressure measurement chamber 1131 and/or a pressure sensor. In reference to FIG. 1a, the pressure measurement chamber 1131 and/or the pressure sensor can comprise an inlet fluidically connected to the inlet 111 and an outlet fluidically connected to the outlet 112, so that the cerebrospinal fluid passes through it. Alternatively or in addition, with reference to FIG. 1b, the pressure measurement chamber 1131 and/or the pressure sensor can comprise an inlet emerging between the inlet 111 and the outlet 112, for example so as to place the pressure measurement chamber 1131 and/or the pressure sensor in bypass relative to the flow element. It is therefore possible to prevent the pressure measurement chamber 1131 and/or the pressure sensor from being subjected to a fluid flow.

The pressure measurement means 113 are, for example, disposed upstream or downstream of the source 120 and the detector 140, and preferably upstream of a valve.

### Implant

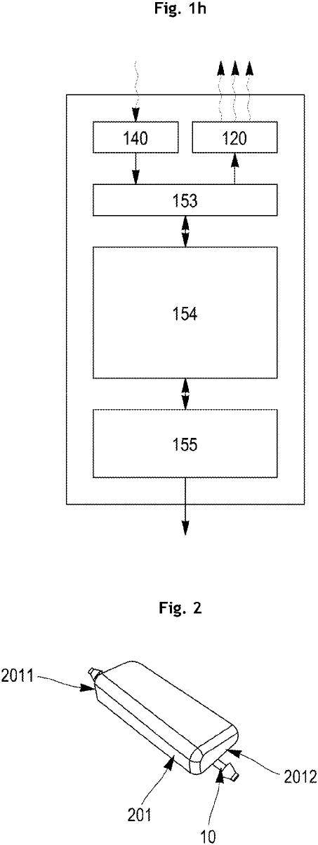

In reference to FIG. 2, an implant 20 is described. The implant 20 can comprise the device 10.

The implant 20 can comprise a housing 201. The device 10 can extend at least partially within housing 201. The housing can comprise an inlet 2011 and an outlet 2012, for example to permit the inlet 111 and the outlet 112 to extend beyond housing 201. The housing can comprise an enclosure, for example a metal enclosure, for example comprising titanium and/or made of titanium. The enclosure can be coated with a layer of silicone, for example implantable silicone.

The implant can be or comprise a catheter valve assembly for diverting a liquid, for example cerebrospinal fluid. The implant can also comprise the characteristics detailed in the application of the patent holder published under number WO2018007574A1.

### Measurement Method

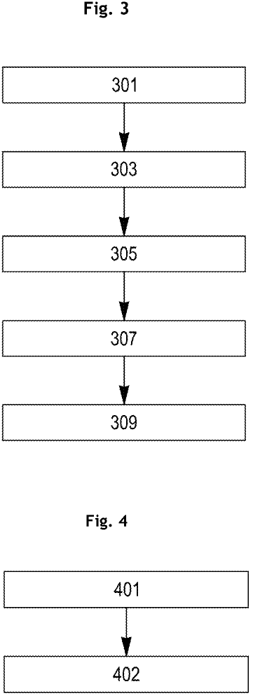

In reference to FIG. 3, a method is described for measuring the turbidity of cerebrospinal fluid by means of the device 10.

The method can comprise a first step 301 for circulating the cerebrospinal fluid through the flow element.

The method can comprise a second step 303 of emitting a light signal by the source.

The method can comprise a third step 305 of reflecting by the flow element 110 and/or the opaque element of the emitted light signal after at least one first passage through the cerebrospinal fluid present in the flow element 110.

The method can comprise a fourth step 307 of detecting, by the detection means 140, of the reflected light signal after at least one second passage through the cerebrospinal fluid present in the flow element 110.

The method can comprise a fifth step 309 of transmitting the data corresponding to the measurement performed in step 304, for example data storage means or data processing means.

### Manufacturing Method

In reference to FIG. 4, a method for manufacturing the device 10 or the implant 20 is described.

The method can comprise a first step 401 of providing or obtaining the flow element.

The method can comprise a second step 402 of obtaining the opaque element, for example at the level of at least a part of the flow element, the obtaining step comprising, for example, injecting coloured pigment of the opaque element around the flow element.

### System

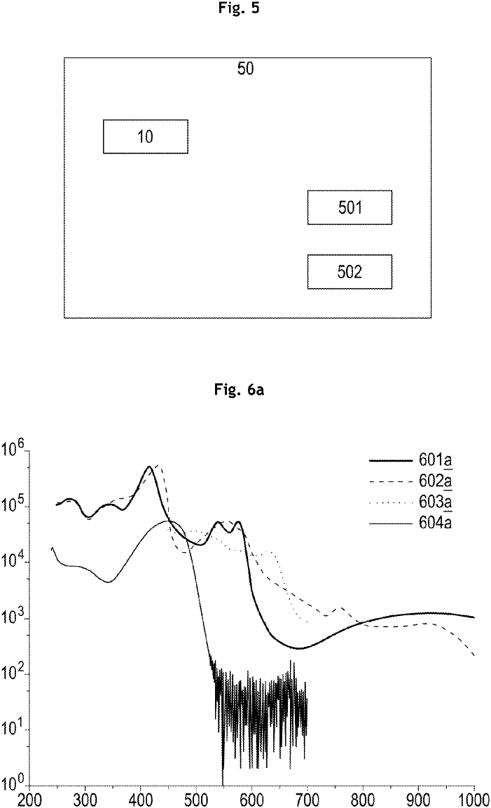

In reference to FIG. 5, a system 50 is described. The system 50 can comprise the device 10 or the implant 20.

The system can comprise the processing means 501 and/or the storage means 502. The processing means 501 are or comprise a processor, for example. The storage means 502 are or comprise a memory, for example.

### Example of Operation

The optical absorption A for the resulting wavelength λ of chromophores in a solution can be defined by the Beer-Lambert law:

$$\text{A} \left(λ\right) = log \left({\text{I}}_{\text{0}} \text{/I}\right) = ε \left(λ\right) .1. \text{c}$$

where A(λ) is absorbance A for wavelength λ
ε(λ) is the molar extinction coefficient of the chromophore in L.mol⁻¹.cm⁻¹
1: the thickness of the liquid crossed by the optical beam in cm
c: the molar concentration of the chromophore in mol/L
This law is verified, for example, when the solution is at a concentration less than 0.1 mol/L.

At a given wavelength λ, absorbance A of a mixture of n absorbing species is the sum of the individual absorbances:

$$A \left(λ\right) = {\sum }_{i = 1}^{N} {A}_{i} \left(λ\right) = {\sum }_{i = 1}^{N} {ε}_{i} \left(λ\right) . l . {c}_{i}$$

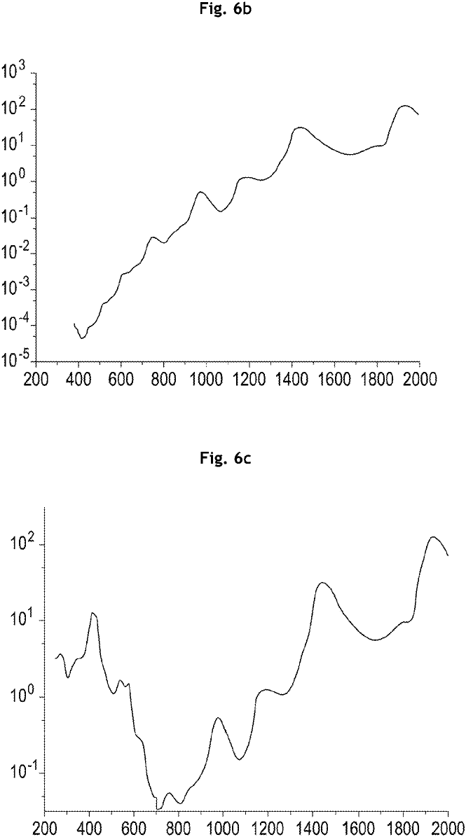

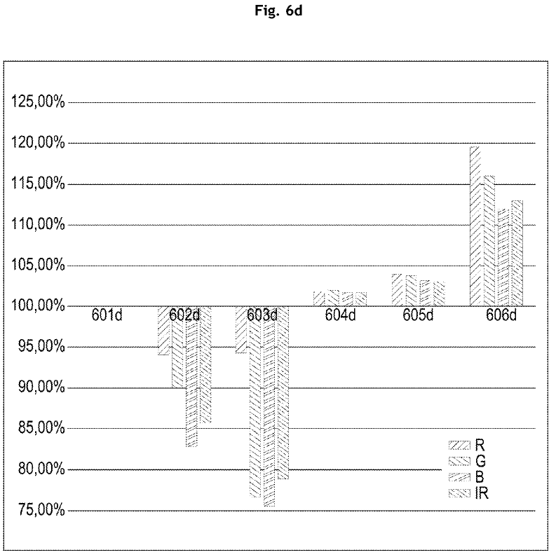

In reference to FIG. 6a, the molar extinction spectrum of the different chromophores is represented. The molar extinction coefficient, for example expressed in L.mol⁻¹.cm⁻¹, for example defined by the Beer-Lambert law, for example applied to one or more absorbing species, can thus be represented for example as a function of the optical wavelength, for example expressed in nm, for different chromophores, for example for oxyhaemoglobin 601a and/or haemoglobin 602a , and/or methaemoglobin 603a and/or bilirubin 604a.

The absorption of such chromophores is typically maximum for wavelengths comprised between 250 and 1000 nm.

In reference to FIG. 6b, the absorption coefficient of water is shown, for example in

cm⁻¹, as a function of the wavelength, for example in nm. For wavelengths between 250 and 1200 nm, the absorption coefficient of water can be lower than the absorption coefficients of several chromophores, for example oxyhaemoglobin and/or haemoglobin, and/or methaemoglobin and/or bilirubin.

In reference to FIG. 6c, the absorption coefficient of a subarachnoid haemorrhage is shown, which forms a cerebrospinal fluid comprising haemoglobin and bilirubin, for example in cm⁻¹, as a function of the wavelength, for example in nm.

The cerebrospinal fluid can comprise water (99%) and/or proteins and/or haemoglobin and/or bilirubin and/or leukocytes, for example depending on the pathological states. For example, the presence of haemoglobin and/or bilirubin can -imply an increase in absorption compared to a healthy fluid, and can therefore characterize a haemorrhagic state. For example, the presence of leukocyte can -imply an increase in diffraction and/or reflection compared to a healthy fluid and can therefore characterize an infectious state. An optical analysis of cerebrospinal fluid is thus possible.

The variation of light intensity received by the measurement means, for example the optical sensors, of the device is shown in reference to FIG. 6d, for example, in four wavelengths, for example red and/or green and/or blue and/or infrared, for example with different liquids presenting different colourations and turbidities, for example pure water 601d and/or yellowish water 602d and/or reddish water 603d and/or whitish water, for example, according to three levels of increasing turbidity 604d, 605d, 606d, compared to pure water taken as reference (100%), for example, in the form of the ratio between the intensity measured for the coloured liquid and the intensity measured for pure water. The light intensity of each component can thus be modified according to the liquid that passes through the device, for example according to the colouration and the turbidity of said liquid which passes through the device.

1. A device for measuring the turbidity of cerebrospinal fluid, comprising:
a source able to emit a light signal comprising one or more wavelengths, such that at least a part of the emitted light signal passes through the cerebrospinal fluid,
a flow element comprising an inlet and an outlet, the flow element being suitable for allowing the cerebrospinal fluid to circulate between the inlet and the outlet,
an opaque element, arranged to absorb at least a part of the emitted light signal after it has passed through the cerebrospinal fluid, and to reflect another part of the emitted light signal after it has passed through the cerebrospinal fluid,
an optical detector configured to detect the light signal after it has passed through the cerebrospinal fluid.

2. The device according to claim 1, wherein the at least one opaque element extends around at least one portion of the flow element.

3. The device according to claim 1, wherein the flow element comprises a wall transparent or translucent to the wavelengths of the light signals emitted by the source, preferably having a transmission coefficient greater than 80%.

4. The device according to claim 1, wherein the flow element and/or the opaque element can comprise an opening opposite which the source and/or the detector are positioned.

5. The device according to claim 1, wherein the light signals emitted by the source comprise one or more wavelength(s) comprised between 300 nm and 850 nm.

6. The device according to claim 1, wherein the source comprises one or more light-emitting diode(s), for example three light-emitting diodes, allowing each one to generate a light signal of wavelength different from the light signal or signals generated by the other diode(s).

7. The device according to claim 1, wherein the detector is designed to measure one or more areas of disjoint wavelengths.

8. The device according to claim 1, wherein the detector is designed to detect a light signal comprising one or more wavelength(s) comprised between 300 nm and 1000 nm.

9. The device according to claim 1, further comprising means for measuring cerebrospinal fluid pressure.

10. An implant comprising a device for measuring the turbidity of cerebrospinal fluid, comprising:
a source able to emit a light signal comprising one or more wavelengths, such that at least a part of the emitted light signal passes through the cerebrospinal fluid,
a flow element comprising an inlet and an outlet, the flow element being suitable for allowing the cerebrospinal fluid to circulate between the inlet and the outlet,
an opaque element, arranged to absorb at least a part of the emitted light signal after it has passed through the cerebrospinal fluid, and to reflect another part of the emitted light signal after it has passed through the cerebrospinal fluid,
an optical detector configured to detect the light signal after it has passed through the cerebrospinal fluid.

11. A method for measuring the turbidity of cerebrospinal fluid by means of an implant comprising a device for measuring the turbidity of cerebrospinal fluid, comprising:
a source able to emit a light signal comprising one or more wavelengths, such that at least a part of the emitted light signal passes through the cerebrospinal fluid,
a flow element comprising an inlet and an outlet, the flow element being suitable for allowing the cerebrospinal fluid to circulate between the inlet and the outlet,
an opaque element, arranged to absorb at least a part of the emitted light signal after it has passed through the cerebrospinal fluid, and to reflect another part of the emitted light signal after it has passed through the cerebrospinal fluid,
an optical detector configured to detect the light signal after it has passed through the cerebrospinal fluid.
comprising the steps of:
circulating the cerebrospinal fluid through the flow element,
emitting a light signal by the source,
reflecting by the flow element and/or the opaque element of the light signal emitted after at least a first passage through the cerebrospinal fluid present in the flow element,
detecting, by the measurement means, of the light signal reflected after at least a second passage through the cerebrospinal fluid present in the flow element.

12. The method for manufacturing a device -for measuring the turbidity of cerebrospinal fluid, comprising:
a source able to emit a light signal comprising one or more wavelengths, such that at least a part of the emitted light signal passes through the cerebrospinal fluid,
a flow element comprising an inlet and an outlet, the flow element being suitable for allowing the cerebrospinal fluid to circulate between the inlet and the outlet,
an opaque element, arranged to absorb at least a part of the emitted light signal after it has passed through the cerebrospinal fluid, and to reflect another part of the emitted light signal after it has passed through the cerebrospinal fluid,
an optical detector configured to detect the light signal after it has passed through the cerebrospinal fluid.
comprising a step of obtaining the opaque element, the obtaining step comprising, for example, injecting coloured pigment of the opaque element around the flow element.
